(19) United States
(12) Patent Application Publication
(10) Pub. No.: US 2006/0173295 A1
(43) Pub. Date: Aug. 3, 2006

(54) Apparatus for detecting strong magnetic fields for protection of medical devices

(76) Inventor: Volkert A. Zeijlemaker, Landgraaf (NL)

Correspondence Address: MEDTRONIC, INC., 710 MEDTRONIC PARK, MINNEAPOLIS, MN, 55432-9924, US

(21) Appl. No.: 11/043,293

(22) Filed: Jan. 26, 2005

(51) Int. Cl. A61B 5/05

(52) U.S. Cl. 600/427

(57) ABSTRACT

A medical device for providing therapy to a host patient comprises a stimulation engine for generating a stimulation regimen and configured to provide the regimen to the patient in a first mode of operation, and a detector coupled to the stimulation engine for detecting the presence of a strong magnetic field and placing the stimulation engine in a second mode of operation in response thereto.

### TECHNICAL FIELD

The present invention relates generally to medical devices, and more particularly to systems and methods of detecting strong magnetic fields in implantable medical devices.

### BACKGROUND

Implantable medical devices (IMDs), such as pacemakers and implantable defibrillators or stimulators utilize sophisticated electronic components, oftentimes in the form of integrated circuits, to minimize device size. It is known, however, that strong magnetic fields may interfere with the proper operation of an IMD or its various associated electronic components, and in some cases, IMDs and/or their associated electronic components are configured to operate in a different mode to offset interference caused by the presence of a strong magnetic field. For example, a pacemaker may include sensing circuitry to detect the strong magnetic fields associated with a magnetic resonance imaging (MRI) system, which typically ranges from about 0.5 Tesla to about 10.0 Tesla.

Reed switches have been used in pacemakers to activate/deactivate the telemetry of the pacemaker. A basic reed switch includes two flattened ferromagnetic reeds that are sealed in a dry inert-gas atmosphere within a glass capsule. The reeds are sealed in the capsule in a cantilever form so that their free ends overlap and are separated by a small air gap. When a magnetic force is applied parallel to the reed switch, the overlapping ends of the reeds become opposite magnetic poles and attract each other. If the magnetic force between the poles is strong enough to overcome the restoring force of the reeds, the reeds are drawn together and the switch is “closed”. Correct alignment of magnetic field lines is generally required for proper operation of a reed switch, and in the presence of a strong magnetic field, such as that which exists in an MRI environment, the reed switch may open when positioned such that the strong magnetic field diminishes the magnetic force applied to the reeds to close the reed switch. Thus, such switches are unreliable as a magnetic field detector in an MRI environment.

Hall effect field sensor devices have been used to detect the presence of a magnetic field. Such sensors are typically characterized by direction sensitivity, and a combination of three such sensors, each being substantially perpendicular to the other sensors, is typically used to detect a magnetic field. The use of three sensors, however, is inconsistent with the goal of producing smaller IMDs that are simpler and less costly. Additionally, such sensors may fail to identify the presence of the magnetic field unless the magnetic field satisfies direction sensitivity (i.e., alignment of the external magnetic field with the sensor).

Additionally, reed switch type sensors and Hall field sensor devices typically operate within a linear region of magnetic field response (e.g., below magnetic field strengths associated with the saturation of their respective magnetic materials) to measure the external magnetic field strength in addition to detecting the presence of the magnetic field. With higher magnetic field strengths, the magnetic materials tend to saturate more quickly which may disrupt such measurements.

Accordingly, it is desirable to provide a simple magnetic field sensing device that may be incorporated into a variety of medical devices. It is also desirable to provide an IMD having a simple magnetic field sensor configuration that alters the operational mode of the IMD upon detection of a strong magnetic field. Furthermore, other desirable features and characteristics of the present invention will become apparent from the subsequent detailed description and the appended claims, taken in conjunction with the accompanying drawings and this background.

### BRIEF SUMMARY

According to various exemplary embodiments, an apparatus is provided to detect the presence of a strong magnetic field in the proximity of a medical device. In an exemplary embodiment, a medical device for providing therapy to a host patient comprises a stimulation engine for generating a stimulation regimen and configured to provide the regimen to the patient in a first mode of operation, and a detector coupled to the stimulation engine for detecting the presence of a strong magnetic field and placing the stimulation engine in a second mode of operation in response thereto.

### BRIEF DESCRIPTION OF THE DRAWINGS

The present invention will hereinafter be described in conjunction with the following drawing figures, wherein like numerals denote like elements, and

FIG. 1 is a block diagram of a medical device including a magnetic field sensor in accordance with an exemplary embodiment of the present invention;

FIG. 2 is a partial schematic diagram of a magnetic field sensor in accordance with a first embodiment of the present invention; and

FIG. 3 is a partial schematic diagram of a magnetic field sensor in accordance with a second embodiment of the present invention.

### DETAILED DESCRIPTION

The following detailed description is merely exemplary in nature and is not intended to limit the invention or the application and uses of the invention. Furthermore, there is no intention to be bound by any expressed or implied theory presented in the preceding technical field, background, brief summary, brief description of the drawings, or the following detailed description.

According to various exemplary embodiments, a sensor for a medical device is provided for detecting the presence of strong magnetic fields, e.g., from about 0.5 Tesla to about 10.0 Tesla. The medical device additionally includes a stimulation engine for generating a stimulation regimen and is configured to provide the regimen to the patient in a first mode of operation. Upon detecting the presence of the strong magnetic field, the sensor places the stimulation engine in a second mode of operation in response thereto. Examples of medical devices include, but are not limited to, implantable medical devices (IMDs) such as neuromuscular stimulators, active IMDs (AIMDs) (e.g., pacemakers and cardiac defibrillators), and drug delivery devices.

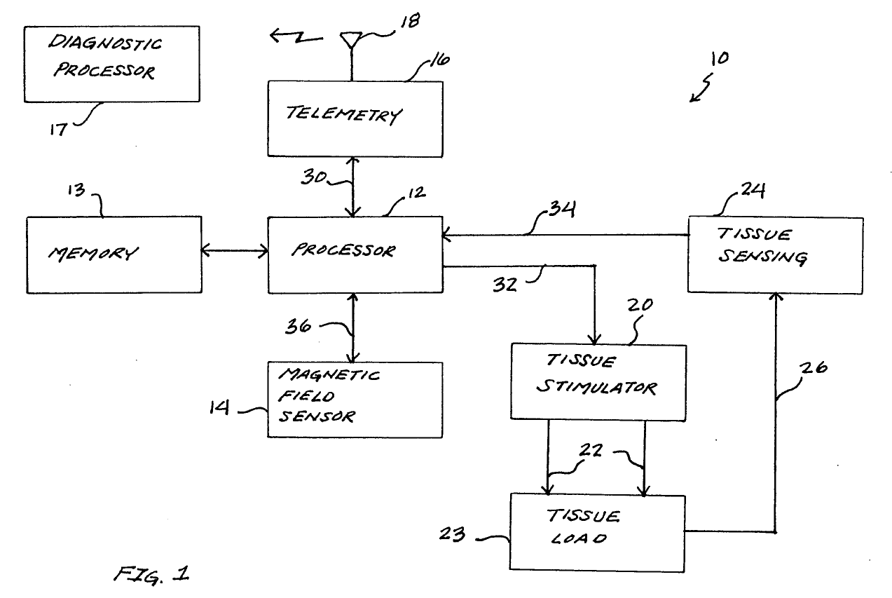

Referring now to the drawings, FIG. 1 is a block diagram of a medical device 10 including a magnetic field sensor 14 in accordance with an exemplary embodiment of the present invention. Medical device 10 comprises a processor 12 for controlling the operation of medical device 10, a telemetry unit 16 coupled to processor 12 via a line 30 for conveying status information to an externally located receiver, such as a diagnostic processor 17, a memory 13 coupled to processor 12 for storing data received by telemetry unit 16 and data to be accessed by processor 12 for controlling the operation of medical device 10, a tissue stimulator circuit 20 including an input coupled to processor 12 via line 32, a lead system 22 coupled to an output of tissue stimulator circuit 20, a tissue sensing circuit coupled to processor 12 via line 34, and magnetic field sensor 14 coupled to processor 12 via line 36. Additionally, processor 12 may receive programming instructions via telemetry unit 16 such as from control signals transmitted via an external transmitter (not shown).

Processor 12 operates via programming instructions that enable the IMD to operate in: (1) a normal mode; and, (2) an alternate mode. The normal mode is appropriate for an environment free of magnetic fields that adversely affect the performance of medical device 10. In such an environment, processor 12 transmits control signals to tissue stimulator circuit 20 to stimulate tissue according to a therapy program that is either pre-determined or based upon feedback from the stimulated tissue. When a strong magnetic field (of the type that occurs during an MRI scan) is detected by magnetic field sensor 14, magnetic field sensor 14 transmits a signal to processor 12 indicating the presence of the strong magnetic field. In response thereto, processor 12 enters the alternate mode of operation and thus prevents therapy corruption that might occur as a result of induced electromagnetic energy. In general, smaller medical devices may be affected to a greater extent by strong magnetic fields due to the use therein of smaller-sized components having more limited operating ranges.

Those of skill in the art will appreciate that IMDs are typically designed to minimize size and operate with less voltage and less current. In the alternate mode of operation, processor 12 may control current and/or voltage sourcing to tissue stimulator 20, and therefore tissue load 23. A variety of other tasks may be performed by processor 12 in the alternate mode to minimize the effects that result from the strong magnetic field.

Tissue stimulator circuit 20 may be configured in a variety of ways depending on the desired stimulation format. In one exemplary embodiment, tissue stimulator circuit 20 is coupled to the target tissue of a patient via lead system 22 comprised of one or more electrodes. For example, tissue stimulator circuit 20 may convey a pacing signal to one or more regions of the heart via the lead system 22 and one or more electrodes positioned in proximity to such regions. In the alternate mode of operation, tissue stimulator circuit 20 may alter the amplitude of the pacing signal. Tissue stimulator circuit 20, as with all other circuits described herein, may comprise various analog and/or discrete digital components as will be appreciated by those of skill in the art.

Tissue sensing circuit 24 detects signals, in the targeted or related tissue (e.g., the cardiac region, spinal region, and the like), related to a physiological response to the tissue stimulation and transmit such signals or an amplified and/or modulated version thereof to processor 12 for analysis. For example, tissue sensing circuit 24 may be connected to a region of a heart via one or more electrodes 26 to monitor local electrical activity and transmit this data to the processor 12. Processor 12 in turn may use this data to control further stimulation of the heart.

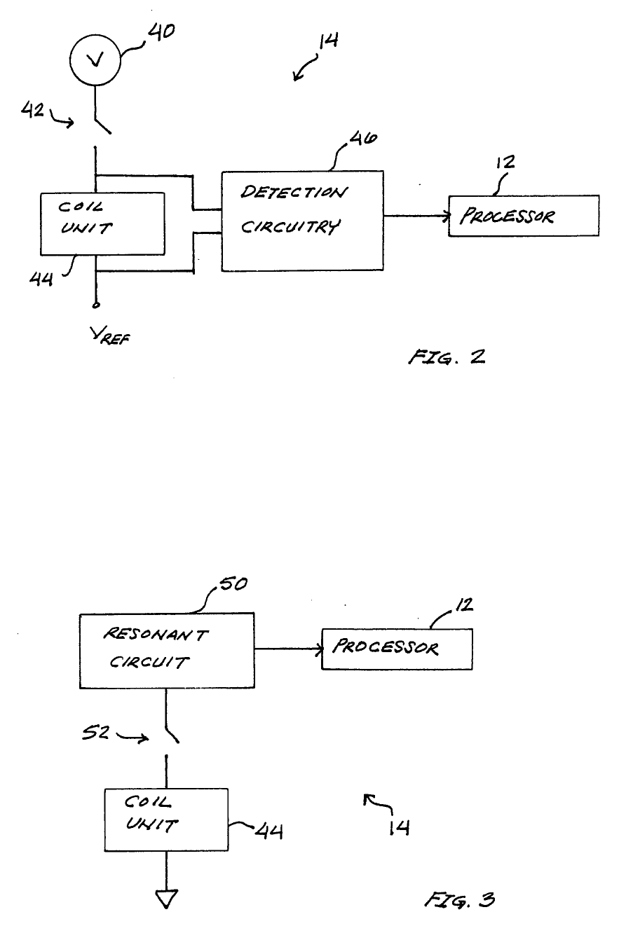

FIG. 2 is a partial schematic diagram of magnetic field sensor 14 in accordance with a first embodiment. The magnetic field sensor 14 comprises a voltage source 40, a switch 42 having a first electrode connected to voltage source 40 and having a second electrode, a coil unit 44 having an input coupled to the second electrode of switch 42 and having an output for coupling to a reference potential, and a detection circuit 46 having a first electrode coupled to the input of coil unit 44 and having a second electrode coupled to the output of coil unit 44. Processor 12 is coupled to an output of detection circuit 46.

In one exemplary embodiment, voltage source 40 intermittently supplies a drive voltage to coil unit 44 via switch 42 to periodically activate magnetic field sensor 14. Coil unit 44 comprises a magnetic material similar to ferrite or the like and is incorporated within the structure of a coil. In one exemplary embodiment, coil unit 44 comprises a core of magnetic material. In another exemplary embodiment, coil unit 44 may be a printed wire coil comprising a layer of magnetic material overlaying the printed wire coil. The coil may be printed onto a silicon substrate, and the magnetic material may be deposited onto the coil using conventional masking and etching techniques. Other coil structures having magnetic properties may also be used.

Examples of ferrites include those with combinations of two or more divalent metal oxides such as zinc, nickel, manganese, and copper. Ferrites have relatively high magnetic permeability (e.g., substantially greater than the permeability of air and typically greater than about 10) and high electrical resistivity which tends to limit electric current flow in ferrites. Magnetic permeability is referred to herein as a relative increase or decrease in the resultant magnetic field inside the magnetic material of coil unit 44 compared with a magnetizing field in which coil unit 44 is located. Detection circuitry 46 measures changes in the magnetic permeability of the magnetic material to detect the presence of a strong magnetic field. When a steady magnetic field is applied to coil unit 44, the relative permeability of the magnetic material is generally reduced, and the inductance associated with the coil unit 44 is also reduced. In this exemplary embodiment, detection circuitry 46 detects the presence of the strong magnetic field by measuring a change in the inductance, associated with strong magnetic fields, that exceeds a threshold.

In one exemplary embodiment, the inductance of coil unit 44 in an environment substantially free of strong magnetic fields is predetermined and stored in memory 13 (FIG. 1) for comparison with intermittently measured inductances of coil unit 44. Processor 12 may also compare one or more recently measured inductances with a currently measured inductance. When processor 12 detects a substantial change (e.g., exceeding a predetermined threshold) in the differences between the measured inductances and the predetermined inductance or in a currently measured inductance from a recently measured inductances, processor 12 enters the alternate mode of operation.

In another exemplary embodiment, detection circuit 42 measures the time for saturation of the magnetic material. Saturation of the magnetic material is generally not dependent on the position of the magnetic material in a strong magnetic field. For example, field strengths encountered in an MRI environment, e.g., above 0.5 Tesla, are sufficient to saturate most ferrite materials independent of the direction of the magnetic field. When voltage source 40 is operatively coupled to coil unit 44, a current passing through coil unit 44 will increase over time to a limit based on the inductance of coil unit 44 generated by voltage source 40, and/or other circuit components as will be appreciated by those of skill in the art. When the inductance is low, such as when the magnetic material becomes saturated in an MRI environment, the current slew rate (e.g., the change in current over time) passing through coil unit 44 is relatively high. By monitoring the slew rate of the current through coil unit 44, the inductance of coil unit 44 may be determined and a substantial change in the slew rate (e.g., exceeding a predetermined threshold of change) may be identified. In this manner, magnetic field sensor 14 can determine when a high magnetic field is present and inform processor 12 to change operation to the alternate mode as described above.

FIG. 3 is a partial schematic diagram of magnetic field sensor 14 in accordance with a second embodiment. Magnetic field sensor 14 comprises coil unit 44, a resonant circuit 50, and a switch 52 having a first electrode coupled to resonant circuit 50 and having a second electrode coupled to the input of coil unit 44. Processor 12 is coupled to an output of resonant circuit 50. The resonant circuit 50 generates a signal having a frequency component, such as a radio frequency (RF) voltage, and excites the magnetic material when operatively coupled with coil unit 44 via switch 52. In response to the signal, coil unit 44 produces a “ringing” waveform that is a function of inductance of the coil and the relative magnetic permeability of the magnetic material. Resonant circuit 50 detects the waveform from coil unit 44, and processor 12 analyzes the waveform characteristics (e.g., frequency and/or amplitude).

When coil unit 44 is in the presence of the strong magnetic field, the relative magnetic permeability of the magnetic material generally diminishes and the ringing frequency generally increases. The frequency of the waveform produced by coil unit 44 in an environment that is substantially free of strong magnetic fields may be predetermined and stored in memory 13 (FIG. 1) for comparison with the frequencies of the waveforms produced during operation of the magnetic field sensor 14. Processor 12 may also compare the frequencies of one or more recently detected waveforms with the frequency of a currently detected waveform. When processor 12 determines a substantial change (e.g., exceeding a predetermined threshold) between the frequencies of the detected waveforms from the predetermined frequency or in the frequency of the currently detected waveform from the frequencies of recently detected waveforms, processor 12 enters the alternate mode of operation.

Thus, magnetic field sensor 14 detects strong magnetic fields (i.e., equal to or greater than about 0.5 Tesla) such as those generated in an MRI environment. Coil unit 44 senses these strong magnetic fields regardless of the direction of such magnetic fields. This permits medical device 10 to be operated in an MRI environment in a mode that compensates for MRI induced field effects.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing the exemplary embodiment or exemplary embodiments. It should be understood that various changes can be made in the function and arrangement of elements without departing from the scope of the invention as set forth in the appended claims and the legal equivalents thereof.

What is claimed is:

1. A medical device for providing therapy to a host patient, the device comprising:
a stimulation engine for generating a stimulation regimen and configured to provide said regimen to the patient in a first mode of operation; and
a detector coupled to said stimulation engine for detecting a presence of a strong magnetic field and placing said stimulation engine in a second mode of operation in response thereto.

2. A medical device according to claim 1, wherein said detector comprises:
a coil unit comprising a magnetic material; and
a processing unit having an input and an output, said input of said processing unit coupled to said coil unit, said output of said processing unit coupled to said stimulation engine, said processor configured to:
detect a change in the magnetic permeability of said magnetic material; and
place said stimulation engine in said second mode of operation when said change in said magnetic permeability exceeds a predetermined threshold indicating the presence of the strong magnetic field.

3. A medical device according to claim 2, wherein said threshold corresponds to a magnetic field equal to or greater than about 0.5 Tesla.

4. A medical device according to claim 1, wherein said detector comprises:
a voltage source configured to intermittently output a voltage;
a coil unit having first and second terminals, said first terminal of said coil unit coupled to said voltage source, said coil unit comprising a magnetic material and configured to generate an inductance based on an external magnetic field; and
a processing unit having first and second inputs and an output, said first input of said processing unit coupled to said first terminal of said coil unit, said second input of said processing unit coupled to said second terminal of said coil unit, and said output of said processing unit coupled to said stimulation engine, said processing unit configured to:
detect a change of said inductance; and
place said stimulation engine in said second mode of operation when said change of said inductance exceeds a predetermined threshold indicating the presence of the strong magnetic field.

5. A medical device according to claim 1, wherein said coil unit comprises a printed wire coil having a layer of said magnetic material.

6. A medical device according to claim 1, wherein said coil unit comprises a core of said magnetic material.

7. A medical device according to claim 1, wherein said detector comprises:
a coil unit comprising a magnetic material having a magnetic permeability;
a resonant circuit coupled to said coil unit, said resonant circuit configured to apply a first signal to said coil unit, said signal having a first frequency, said coil unit configured to generate a second signal in response to said first signal, said second signal having a second frequency based on said magnetic permeability; and
a processor coupled with said resonant circuit and configured to:
detect a frequency change between said second signal from said coil unit and said first signal from said resonant circuit; and
place said stimulation engine in said second mode of operation when said frequency change exceeds a threshold corresponding to the presence of the strong magnetic field.

8. A medical device according to claim 1, wherein said detector comprises:
a voltage source having a current limit;
a coil unit having first and second terminals, said first terminal of said coil unit coupled to said voltage source, said coil unit comprising a magnetic material having a magnetic permeability, said coil unit configured to pass a current therethrough at a slew rate based on said current limit; and
a processor coupled across said first and second terminals of said coil unit and further coupled to said voltage source, said processor configured to:
detect a change in said slew rate; and
place said stimulation engine in said second mode of operation when said change in said slew rate exceeds a predetermined threshold indicating the presence of the strong magnetic field.

9. A medical device according to claim 1, wherein said stimulation engine comprises a pacemaker.

10. A medical device according to claim 1, wherein said stimulation engine comprises an implantable defibrillator.

11. A medical device according to claim 1, wherein said stimulation engine comprises a neurostimulator.

12. An implantable medical device (IMD) for providing therapy to a host patient, the IMD comprising:
a lead system configured for coupling to the host patient;
a stimulation engine coupled to said lead system for generating a stimulation regimen, said stimulation engine configured to provide said regimen to the host patient via said lead system in a first mode of operation; and
a detector coupled to said stimulation engine for detecting a presence of a strong magnetic field and placing said stimulation engine in a second mode of operation in response thereto.

13. An IMD according to claim 12, wherein said stimulation engine transmits a first signal to said lead system in said first mode of operation and transmits a second signal to said lead system in said second mode of operation.

14. An IMD according to claim 13, wherein said detector comprises:
a coil unit comprising a magnetic material; and
a processing unit having an input and an output, said input of said processing unit coupled to said coil unit, said output of said processing unit coupled to said stimulation engine, said processor configured to:
intermittently measure a magnetic permeability of said magnetic material;
detect a change in said magnetic permeability; and
place said stimulation engine in said second mode of operation when said change in said magnetic permeability exceeds a predetermined threshold indicating the presence of the strong magnetic field.

15. An IMD according to claim 13, wherein said threshold corresponds to a magnetic field equal to or greater than about 0.5 Tesla.

16. An IMD according to claim 13, wherein said detector comprises:
a voltage source configured to intermittently output a voltage;
a coil unit having first and second terminals, said first terminal of said coil unit coupled to said voltage source, said coil unit comprising a magnetic material and configured to generate an inductance based on an external magnetic field; and
a processing unit having first and second inputs and an output, said first input of said processing unit coupled to said first terminal of said coil unit, said second input of said processing unit coupled to said second terminal of said coil unit, and said output of said processing unit coupled to said stimulation engine, said processing unit configured to:
detect a change of said inductance; and
place said stimulation engine in said second mode of operation when said change of said inductance exceeds a predetermined threshold indicating the presence of the strong magnetic field.

17. An IMD according to claim 13, wherein said coil unit comprises a printed wire coil having a layer of said magnetic material.

18. An IMD according to claim 13, wherein said coil unit comprises a core of said magnetic material.

19. An IMD according to claim 13, wherein said detector comprises:
a coil unit comprising a magnetic material having a magnetic permeability;
a resonant circuit coupled to said coil unit, said resonant circuit configured to apply a first signal to said coil unit, said signal having a first frequency, said coil unit configured to generate a second signal in response to said first signal, said second signal having a second frequency based on said magnetic permeability; and
a processor coupled with said resonant circuit and configured to:
detect a frequency change between said second signal from said coil unit and said first signal from said resonant circuit; and
place said stimulation engine in said second mode of operation when said frequency change exceeds a predetermined threshold indicating the presence of the strong magnetic field.

20. An IMD according to claim 12, wherein said detector comprises:
a voltage source having a current limit;
a coil unit having first and second terminals, said first terminal of said coil unit coupled to said voltage source, said coil unit comprising a magnetic material having a magnetic permeability, said coil unit configured to pass a current therethrough at a slew rate based on said current limit; and
a processor coupled across said first and second terminals of said coil unit and further coupled to said voltage source, said processor configured to:
detect a slew rate change; and
place said stimulation engine in said second mode of operation when said slew rate change exceeds a predetermined threshold indicating the presence of the strong magnetic field.
